(19) United States
(12) Patent Application Publication
(10) Pub. No.: US 2026/0069575 A1
(43) Pub. Date: Mar. 12, 2026

(54) USE OF APICIDIN COMBINED WITH BEVACIZUMAB IN PREPARING DRUG FOR TREATING CERVICAL CANCER

(71) Applicants: The First Affiliated Hospital of Xinxiang Medical University, Weihui (CN); Xinxiang Medical University, Weihui (CN)

(72) Inventors: Jiateng ZHONG, Weihui (CN); Wenyu DI, Weihui (CN); Wei SU, Weihui (CN); Miaomiao HAN, Weihui (CN); Yiyang CHEN, Weihui (CN); Xin DONG, Weihui (CN); Yunjuan JIAO, Weihui (CN)

(73) Assignees: The First Affiliated Hospital of Xinxiang Medical University, Weihui (CN); Xinxiang Medical University, Weihui (CN)

(21) Appl. No.: 19/321,380

(22) Filed: Sep. 8, 2025

(30) Foreign Application Priority Data
Sep. 9, 2024 (CN) 202411256834.9

(51) Int. Cl. A61K 31/4353; A61K 39/00; A61K 39/395; A61P 35/00

(52) U.S. Cl. CPC A61K 31/4353; A61K 39/3955; A61P 35/00; A61K 2039/505

(57) ABSTRACT

A use of Apicidin combined with Bevacizumab in preparing a drug for treating cervical cancer is provided. Studies have shown that Apicidin increases the sensitivity of cervical cancer cells to the targeted drug Bevacizumab. The combined use of Apicidin and Bevacizumab can more effectively inhibit the growth of cervical cancer cells and enhance the ability to induce apoptosis of cervical cancer cells, showing a good synergistic effect. Therefore, the use prospects of Apicidin combined with the targeted drug Bevacizumab are huge, which provides a more effective and feasible solution for the clinical treatment of cervical cancer, and proposes a new theoretical basis for the combined treatment of clinical cervical cancer recurrence and drug resistance.

### CROSS REFERENCE TO THE RELATED APPLICATIONS

This application is based upon and claims priority to Chinese Patent Application No. 202411256834.9, filed on Sep. 9, 2024, the entire contents of which are incorporated herein by reference.

### TECHNICAL FIELD

The present invention relates to the technical field of anti-tumor drugs, and in particular, to use of Apicidin combined with Bevacizumab in preparing a drug for treating cervical cancer.

### BACKGROUND

Cervical cancer (CC) is one of the most common malignant tumors in human cancers and a huge challenge facing women worldwide, which is usually caused by human papillomavirus (HPV) infection. Although cervical cancer screening and HPV vaccination have been widely used, a large number of cases still exist. Surgery, radiotherapy and chemotherapy are currently the main methods for treating cervical cancer. However, the high incidence of tumor recurrence and disease progression after these conventional therapies are of great concern and require the development of new therapies. Furthermore, these traditional monotherapies non-selectively target actively proliferating cells, ultimately leading to the simultaneous destruction of healthy and cancerous cells. Instead, combination therapy, which combines 2 therapeutic agents, has become the cornerstone of cancer treatment. By targeting different pathways, combination therapy may reduce the incidence of cancer cell resistance and may reduce toxicity by reducing the required doses of single drugs.

Apicidin is a cyclic peptide histone deacetylase (HDAC) inhibitor that has been shown to exhibit antitumor activity in multiple human cancer types. Acetylation and deacetylation of nucleosome core histones play an important role in the regulation of chromatin structure and gene transcription. Histone acetylation is controlled by the activities of two enzyme families: histone acetyltransferase (HAT) and histone deacetylase (HDAC). Imbalance in histone acetylation status leads to abnormal cell behavior in morphology, cell cycle, differentiation and carcinogenesis. In recent years, an increasing number of structurally diverse HDAC inhibitors have been discovered, including trichostatin A, trichotoxin, sodium butyrate, and the like, which inhibit cell proliferation by blocking the cell cycle, induce differentiation and morphological changes in oncogene-transformed cells, and exhibit anti-tumor activity in vivo. Furthermore, Apicidin has been reported to possess anti-angiogenic potential both in vitro and in vivo, indicating that Apicidin indirectly inhibits the hypoxia-induced angiogenesis mediator vascular endothelial growth factor (VEGF) and suppresses the migration and proliferation of endothelial cells.

Bevacizumab is an anti-angiogenic drug that targets vascular endothelial growth factor (VEGF-A), which mainly inhibits vascular endothelial growth factor (VEGF) from activating cell surface tyrosine kinase receptors (VEGFR-1 and VEGFR-2), generates downstream signals to reduce endothelial cell proliferation and migration, immature endothelial cell growth, increase vascular permeability, and ultimately inhibits the formation of new blood vessels. However, angiogenesis is also associated with immunosuppression; therefore, angiogenesis and immunosuppression may occur simultaneously during tumor formation and progression. In fact, a plurality of pro-angiogenic factors, especially vascular endothelial growth factor (VEGF-A), the main stimulant of angiogenesis, have immunosuppressive functions. Therefore, targeting angiogenesis pathways has been used to restore anti-tumor immune responses, which plays a very important role in inhibiting tumor growth.

Studies have shown that Bevacizumab may not be as effective as expected in some patients with cervical cancer, possibly because of the development of drug resistance.

### SUMMARY

To resolve the recurrence and drug resistance in patients with cervical cancer, the present invention provides the following technical solutions.

The present invention further provides use of Apicidin combined with Bevacizumab in preparing a drug for treating cervical cancer.

Preferably, a molar ratio of the combined use of Apicidin and Bevacizumab is (10-40): (10-320).

Preferably, a cancer cell of the cervical cancer is Hela, Siha or U14.

Preferably, the drug induces apoptosis of Hela, Siha or U14.

The above studies show that Apicidin increases the sensitivity of cervical cancer cells to the targeted drug Bevacizumab. The combined use of Apicidin and Bevacizumab can more effectively inhibit the growth of cervical cancer cells and enhance the ability to induce apoptosis of cervical cancer cells, showing a good synergistic effect. Therefore, the use prospects of Apicidin combined with the targeted drug Bevacizumab are huge, which provides a more effective and feasible solution for the clinical treatment of cervical cancer, and proposes a new theoretical basis for the combined treatment of clinical cervical cancer recurrence and drug resistance.

### BRIEF DESCRIPTION OF THE DRAWINGS

FIG. 1 shows the effects of different concentrations of Apicidin alone on the viability of human cervical cancer cells Hela and Siha as well as mouse cervical cancer cells U14.

FIG. 2 shows the effects of different concentrations of Apicidin alone on the proliferation rates of human cervical cancer cells Hela and Siha as well as mouse cervical cancer cells U14.

FIG. 3 shows the photos of the proliferation of human cervical cancer cells Hela and Siha as well as mouse cervical cancer cells U14 using different concentrations of Apicidin alone.

FIG. 4 shows the photos of the migration of human cervical cancer cells Hela and Siha as well as mouse cervical cancer cells U14 using different concentrations of Apicidin alone.

FIG. 5 shows the expression of related apoptosis proteins in human cervical cancer cells Hela and Siha as well as mouse cervical cancer cells U14 induced by using different concentrations of Apicidin alone.

FIGS. 6A-6L show the apoptosis of human cervical cancer cells Hela and Siha as well as mouse cervical cancer cells U14 induced by using different concentrations of Apicidin alone.

FIG. 7 shows the effects of Apicidin combined with the targeted drug Bevacizumab on the viability of human cervical cancer cells Hela and Siha as well as mouse cervical cancer cells U14.

FIG. 8 shows the effects of Apicidin combined with the targeted drug Bevacizumab on the proliferation rates of human cervical cancer cells Hela and Siha as well as mouse cervical cancer cells U14.

FIG. 9 shows the photos of the proliferation of human cervical cancer cells Hela and Siha as well as mouse cervical cancer cells U14 treated with Apicidin combined with the targeted drug Bevacizumab.

FIG. 10 shows the photos of the migration of human cervical cancer cells Hela and Siha as well as mouse cervical cancer cells U14 treated with Apicidin combined with the targeted drug Bevacizumab.

FIG. 11 shows that the expression of related apoptosis proteins in cervical cancer cells Hela and Siha as well as mouse cervical cancer cell U14 enhanced and induced by Apicidin combined with the targeted drug Bevacizumab.

FIGS. 12A-12L show that apoptosis of cervical cancer cells Hela and Siha as well as mouse cervical cancer cell U14 enhanced and induced by Apicidin combined with the targeted drug Bevacizumab.

FIG. 13 shows the changes in subcutaneous tumor size in nude mice modeled with mouse cervical cancer cell U14 after treatment with Apicidin combined with the targeted drug Bevacizumab.

FIG. 14 shows the statistical results of the changes in subcutaneous tumor size in nude mice modeled with mouse cervical cancer cell U14 after treatment with Apicidin combined with the targeted drug Bevacizumab.

FIG. 15 shows the changes in body weight of nude mice modeled with mouse cervical cancer cell U14 after treatment with Apicidin combined with the targeted drug Bevacizumab.

FIG. 16 shows the HE staining results of tumor tissues in nude mice modeled with mouse cervical cancer cell U14 after treatment with Apicidin combined with the targeted drug Bevacizumab.

FIG. 17 shows the Ki67 staining results of tumor tissues in nude mice modeled with mouse cervical cancer cell U14 after treatment with Apicidin combined with the targeted drug Bevacizumab.

### DETAILED DESCRIPTION OF THE EMBODIMENTS

The technical solutions provided by the present invention will be described in detail below with reference to examples, which, however, should not be construed as limiting the scope of the present invention.

The mouse cervical cancer cell line U14 required for the following examples was purchased from the Delf Cell Bank (hfwanwu, Hefei, China), and the human cervical cancer cells Hela and Siha were purchased from Procell (Wuhan, China). The culture medium, PBS, and trypsin used were purchased from Wuhan Servicebio Technology Co., Ltd. Fetal bovine serum trypsin was purchased from Gibco, USA. 1% streptomycin/penicillin was purchased from Beyotime Pharmaceuticals, China. The drugs used, Apicidin and Bevacizumab, were purchased from MedChemExpress (MCE, USA) in the United States and diluted according to the drug instructions. The antibodies used, Cleaved-caspase-3 (GB11767C-100); Bax (GB12690-100); and Bcl-2 (GB124830-100), were purchased from Wuhan Servicebio Technology Co., Ltd. and diluted according to the instructions. The apoptosis kit was purchased from Dohjin Co., Ltd. (Japan). The drugs, reagents, antibodies, and the like used in the experiment may be purchased from other commercial sources. The experimental results are only used to illustrate the present invention and are not used to limit the scope of the present invention. The experimental method was performed according to routine operating procedures or the experimental method provided by the reagent manufacturer.

Cell culture: Human cervical cancer cells Hela and Siha and mouse cervical cancer cells U14 were cultured in a DMEM medium containing 10% fetal bovine serum and 1% streptomycin/penicillin. The cell culture conditions were: 37° C., 5% CO₂ in an incubator.

### Example 1

In Vitro Experiments

### 1. Effects of Different Concentrations of Apicidin on the Viability and Migration of Cervical Cancer Cells Hela, Siha, and U14

Hela, Siha and U14 cells in the logarithmic growth phase and good condition were selected to plate 96-well plates. 1×10⁴ cells per well were used for survival assay, and 0.2×10⁴ cells/well were used for proliferation assay. Five replicate wells were set for each drug concentration. The concentration gradient of Apicidin diluted with the DMEM medium was: 0, 5, 10, 20, 30, 40 μM.

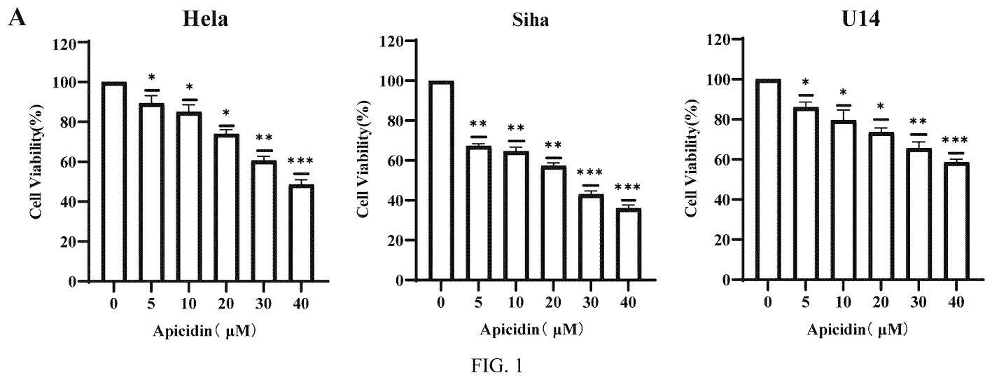

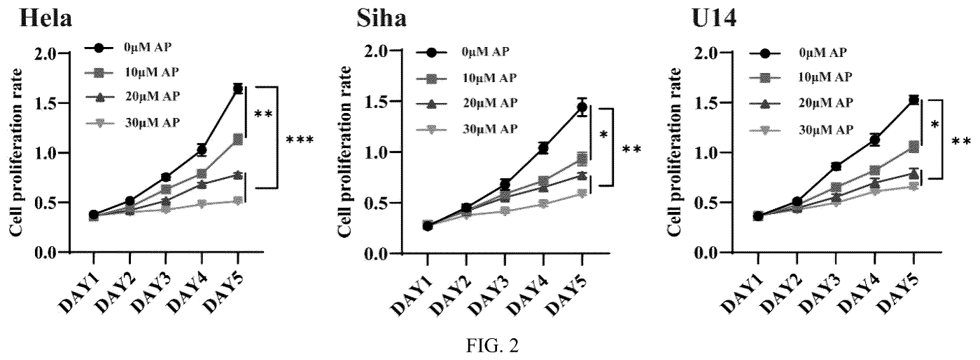

The specific steps were as follows: the original culture medium was discarded, the cells were washed twice with 3 mL of PBS, the cells were digested with 1 mL of trypsin for 1 min, 2 mL of culture medium was added to neutralize the trypsin, and the cell suspension was transferred to a centrifuge tube and centrifuged at 2000 rpm for 1.5 min. The supernatant was discarded, new culture medium was added to resuspend the cells, and 10 μL was taken for counting to calculate the required cell suspension. The 96-well plate was plated in 100 μL/well. After 24 h of cell attachment, Apicidin was diluted to 100 μL containing drug concentrations of 0, 5, 10, 20, 30, and 40 μM. After 24 h of drug treatment, 10 μL of CCK8 was added and incubated in a 37° C. incubator for 2 h. The absorbance was measured at 450 nm using a microplate reader. The cell viability is shown in FIG. 1. Similarly, 0.2×10⁴ cells/well were plated in a 96-well plate with 100 μL of culture medium per well. After 24 h of cell attachment, Apicidin was diluted to 100 μL containing drug concentrations of 0, 10, 20, and 30 μM, and 10 μL of CCK-8 solution was added to each well on days 1-5. After incubation at 37° C. for 2 h, the absorbance was measured at 450 nm using a microplate reader. The cell proliferation rate is shown in FIG. 2. The results in FIGS. 1 and 2 show that the viability and proliferation rate of Hela, Siha, and U14 cells decrease with the increase of drug concentration, which suggests that Apicidin may inhibit the growth of Hela, Siha and U14 cells.

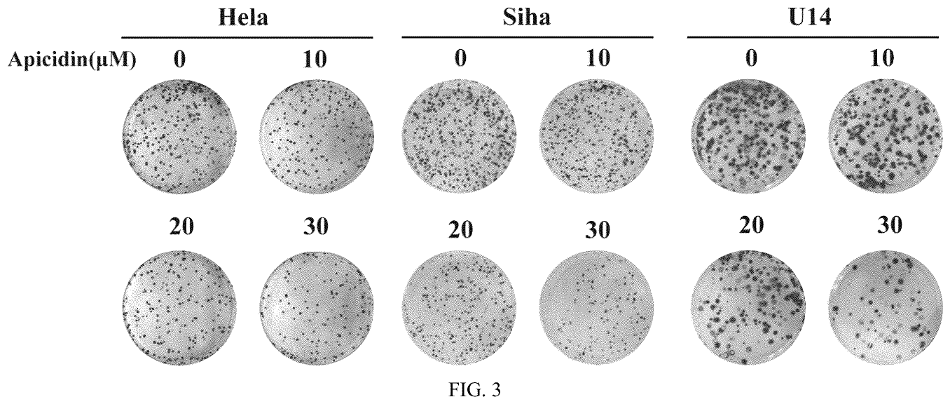

Hela, Siha, and U14 cells in the logarithmic growth phase were seeded in 6-well plates at 0.05×10⁴ cells/well. One week later, the cells were incubated with different concentrations of Apicidin (0, 10, 20, 30 μM) for 7 days. The formation of punctate colonies at the bottom of the 6-well plates was observed, and the cells were fixed with 4% tissue fixative, stained with 1% crystal violet for 30 minutes, and photographed for analysis. The results in FIG. 3 show that Apicidin can inhibit the proliferation of cervical cancer cells Hela, Siha, and U14 with the increase of the drug concentration.

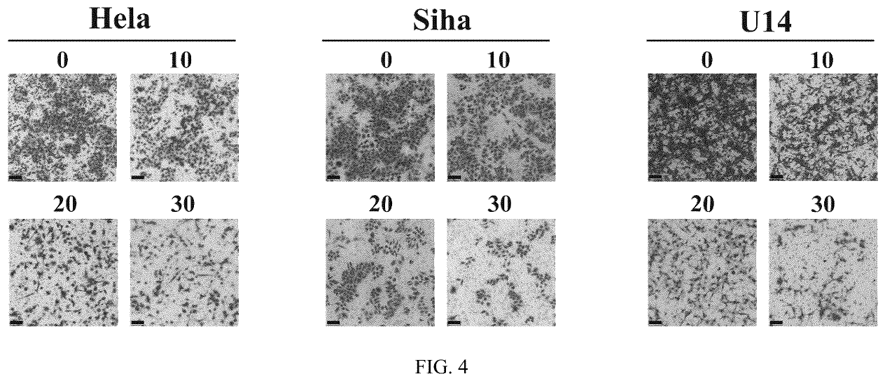

The cells were suspended in serum-free medium, adjusted to a cell concentration of 5×10⁴ cells/well, and inoculated into the upper chamber of the Transwell chamber, and serum-containing medium was added to the lower chamber. Apicidin at different concentrations (0, 10, 20, 30 μM) was added and incubated with the cells for 24 h. The upper chamber was fixed with 4% tissue fixative and stained with crystal violet for 30 min. The interior of the upper chamber was wiped with a cotton swab, and the cells were observed and photographed under a microscope for counting and analysis. The results in FIG. 4 show that Apicidin can inhibit the migration of cervical cancer cells Hela, Siha, and U14 with the increase of the drug concentration.

### 2. Effect of Apicidin on Apoptosis of Cervical Cancer Cells Hela, Siha, and U14

Hela, Siha, and U14 cells in the logarithmic growth phase were digested and centrifuged. After being resuspended, the cells were seeded into 6 cm culture dishes and treated with drugs 24 hours after the cells were completely attached. The drug concentrations of Apicidin were 0, 10, 20, and 30 μM. After 18 hours of action, the next step was performed.

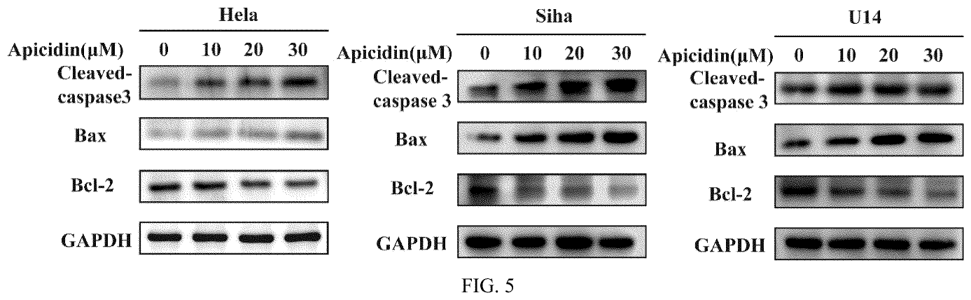

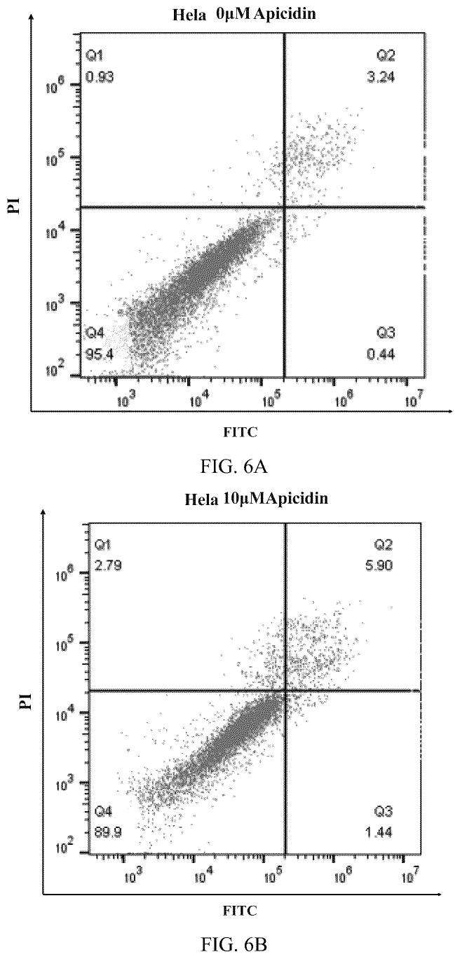

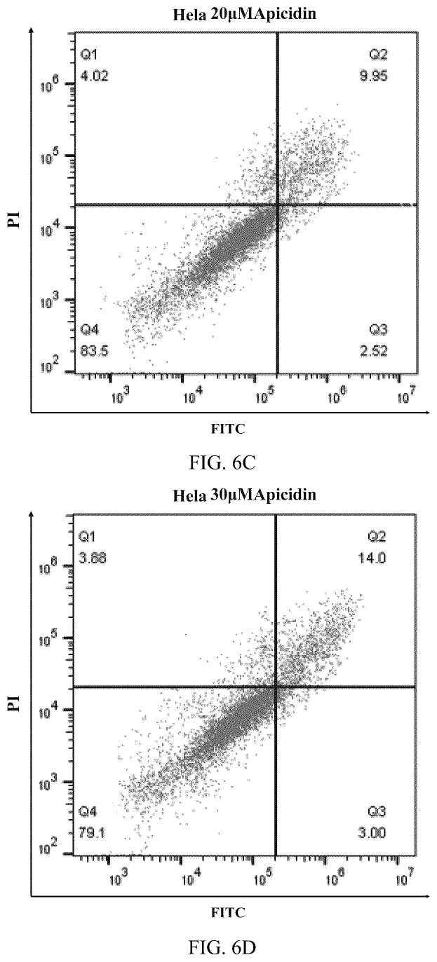

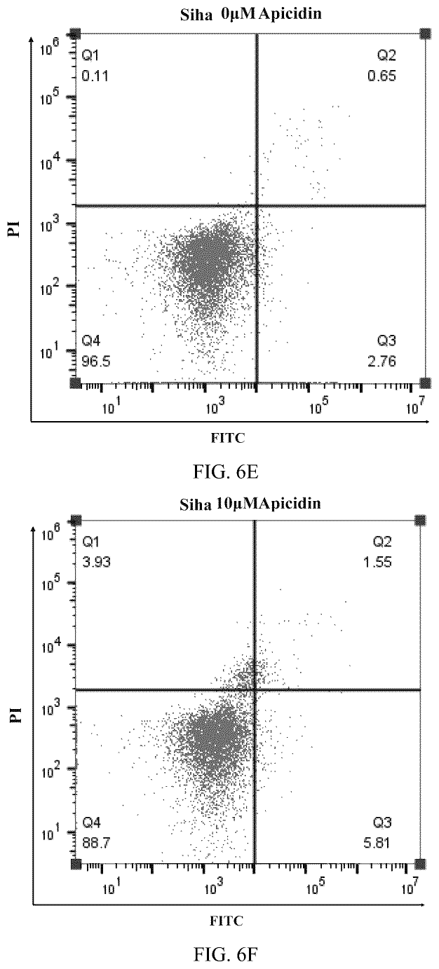

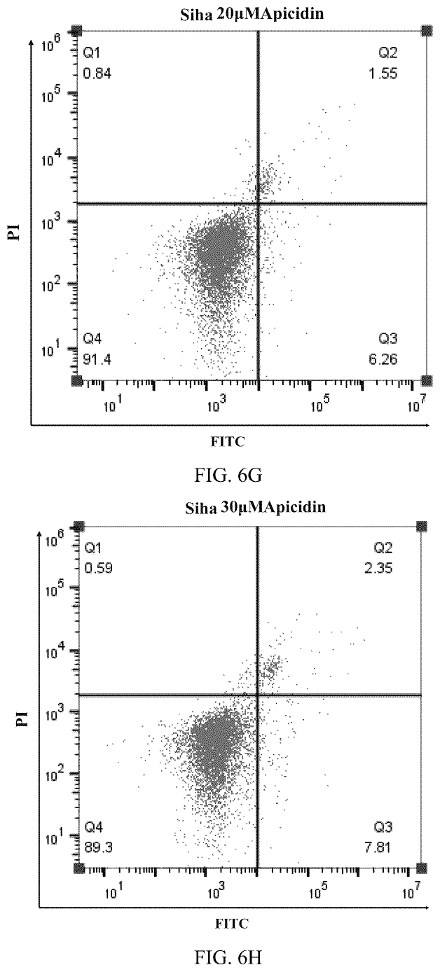

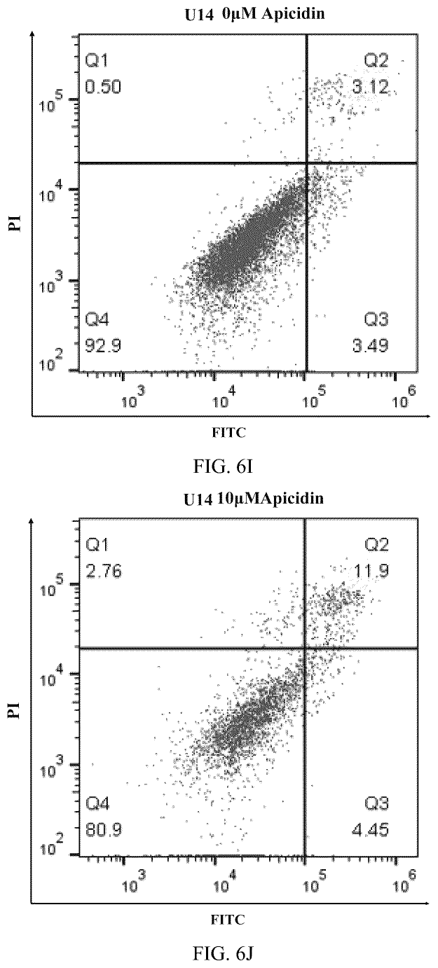

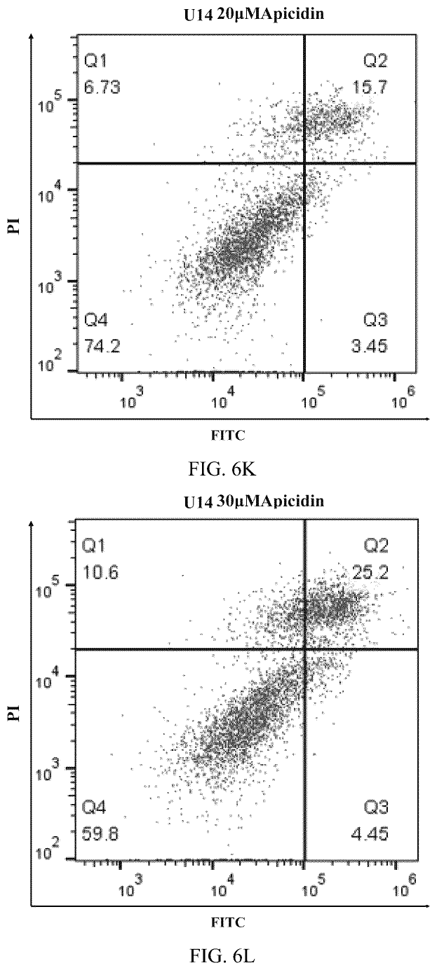

Protein extraction: After drug treatment, the supernatant of the cells was discarded, and the cells were washed twice with PBS. An appropriate amount of cell lysis buffer was added, and the cells were lysed on ice for 5 min. The cells were scraped and the lysate was transferred to an EP tube. After lysis on ice for 20 min, the cells were centrifuged at 4° C. and 12000 rpm for 20 minutes, and the supernatant was collected. After the protein concentration was measured in the BCA working method, the supernatant was diluted to 1× by adding 5× protein loading buffer, boiled in a metal bath at 100° C. for 10 min, and then loaded. According to the Western blotting experimental procedures, the expression of apoptosis-related Cleaved-caspase-3, Bax, and Bcl-2 was detected. Apoptosis detection: According to the apoptosis kit operating instructions, 10× Annexin V binding buffer was diluted to 1× Annexin V binding buffer. After drug treatment, the supernatant of the cells was discarded, and the cells were washed twice with PBS, trypsinized, and centrifuged. The supernatant was discarded, and 1 mL of 1× Annexin V binding Buffer was added to resuspend the cells and centrifuged. The supernatant was discarded, and the above steps were repeated once. 400 μL of 1× Annexin V binding Buffer was added to resuspend the cells. Then, 5 μL of FITC/PI reagent was added, and the cells were protected from light for 15 min before detection. The results are shown in FIG. 5 and FIGS. 6A-6L. As the drug concentration increases, the expression levels of apoptosis-related proteins Cleaved-caspase-3 and Bax in Hela, Siha, and U14 cells increase, the expression level of Bcl-2 decreases, and the number of apoptotic cells increases. These results suggest that Apicidin can induce apoptosis in Hela, Siha and U14 cells.

3. Effects of Apicidin Combined with Targeted Drug Bevacizumab on the Viability and Migration of Cervical Cancer Cells Hela, Siha, and U14

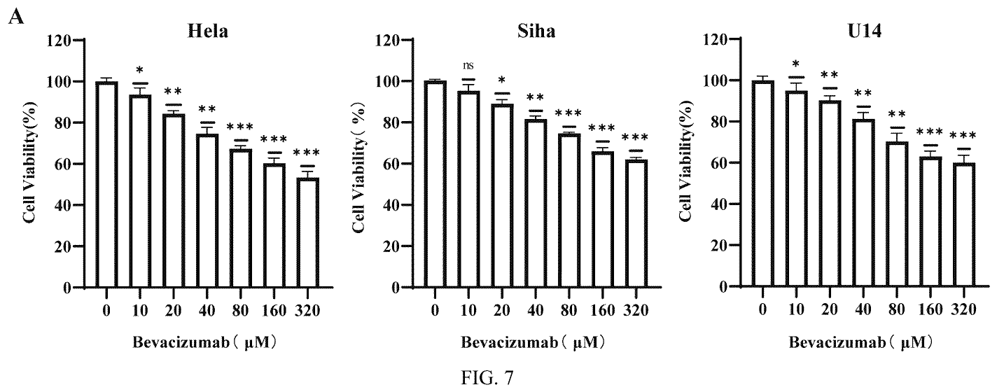

Hela, Siha and U14 cells in the logarithmic growth phase and with good condition were selected to plate 96-well plates, with approximately 1×10⁴ cells/well used for survival assay, and 5 replicate wells were set for each drug concentration. Bevacizumab alone at different concentrations (0, 10, 20, 40, 80, 160, and 320 μM) may reduce the viability of cervical cancer cells Hela, Siha, and U14 (as shown in FIG. 7).

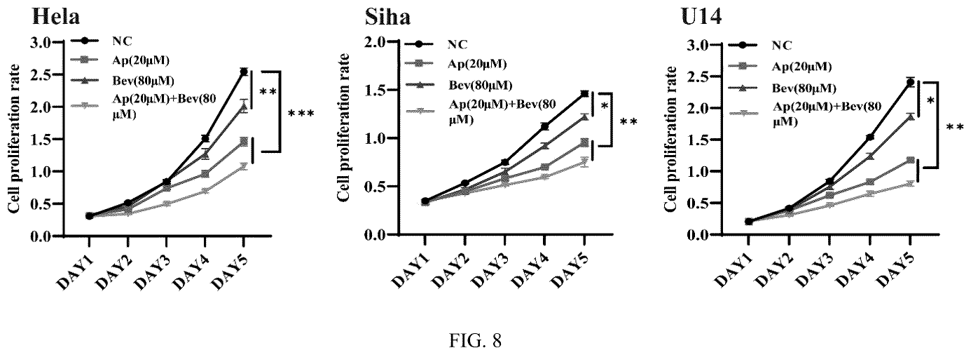

The final concentration of Apicidin was 20 μM, and the final concentration of Bevacizumab was 80 μM. 100 μL of drug-containing culture medium was added to each well. The order of drug addition was: blank (NC), Apicidin (Ap), Bevacizumab (Bev), and Apicidin+Bevacizumab (Ap+Bev). The specific steps were as follows: the original culture medium was discarded, the cells were washed twice with 3 mL of PBS, the cells were digested with 1 mL of trypsin for 1 min, 2 mL of culture medium was added to neutralize the trypsin, and the cell suspension was transferred to a centrifuge tube and centrifuged at 2000 rpm for 1.5 min. The supernatant was discarded, new culture medium was added to resuspend the cells, and 10 μL was taken for counting to calculate the required cell suspension. 0.2×10⁴ cells/well were plated in 96-well plates with 100 μL of culture medium per well, for a total of 4 drug concentrations, and 5 replicate wells were set for each drug concentration. After 24 hours of cell attachment, diluted drug concentrations were added. On days 1-5, 10 μL of CCK-8 solution was added to each well and incubated at 37° C. for 2 h. The absorbance was measured at 450 nm using a microplate reader, as shown in FIG. 8.

As shown in FIGS. 7 and 8, compared with the groups treated with either drug alone, the growth of Hela, Siha, and U14 cells was significantly inhibited after treatment with Apicidin combined with Bevacizumab. The results suggest that Apicidin can enhance the ability of the targeted drug Bevacizumab to inhibit the growth of Hela, Siha, and U14 cells.

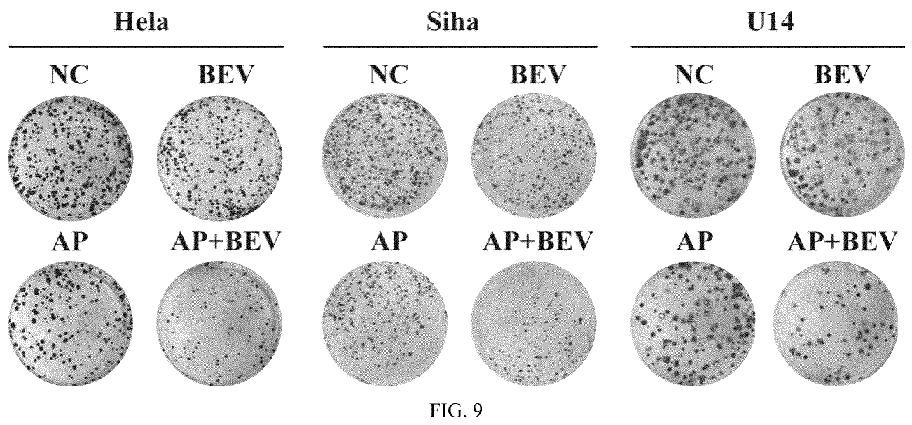

Hela, Siha, and U14 cells in the logarithmic growth phase were seeded in 6-well plates at 0.05×10⁴ cells/well. One week later, Apicidin and Bevacizumab were co-incubated with the cells alone or in combination for 7 days. The formation of punctate colonies at the bottom of the 6-well plates was observed. The cells were fixed with 4% tissue fixative, stained with 1% crystal violet for 30 min, and photographed for analysis. As shown in FIG. 9, the combined administration of Apicidin and Bevacizumab may significantly inhibit the proliferation of cervical cancer cells.

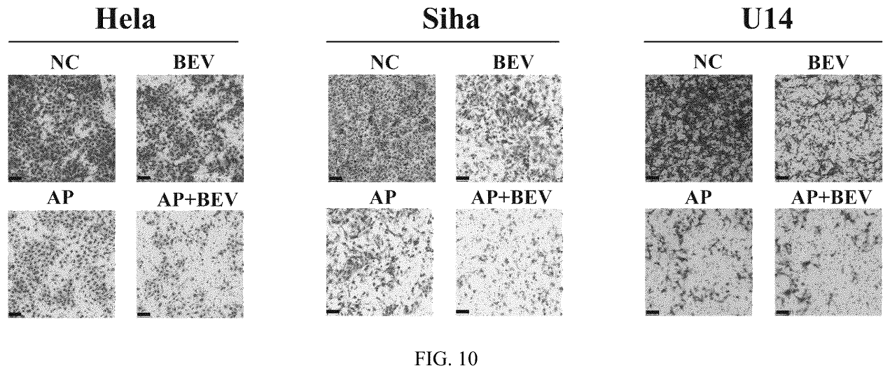

The cells were suspended in serum-free medium, adjusted to a cell concentration of 5×10⁴ cells/well, and inoculated into the upper chamber of the Transwell chamber, and serum-containing medium was added to the lower chamber. Apicidin and Bevacizumab were added alone or in combination and incubated with the cells for 24 h. The upper chamber was fixed with 4% tissue fixative and stained with crystal violet for 30 min. The interior of the upper chamber was wiped with a cotton swab, and the cells were observed and photographed under a microscope for counting and analysis. As shown in the results of FIG. 10, the combined administration of Apicidin and Bevacizumab more Significantly inhibit the migration of cervical cancer cells.

4. Effect of Apicidin combined with Targeted Drug Bevacizumab on Apoptosis of Cervical Cancer Cells Hela, Siha, and U14

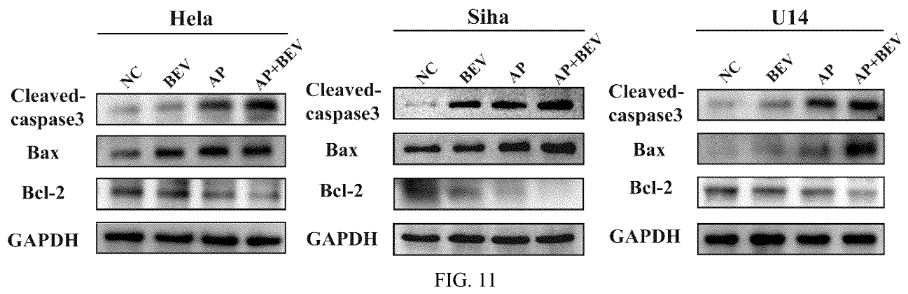

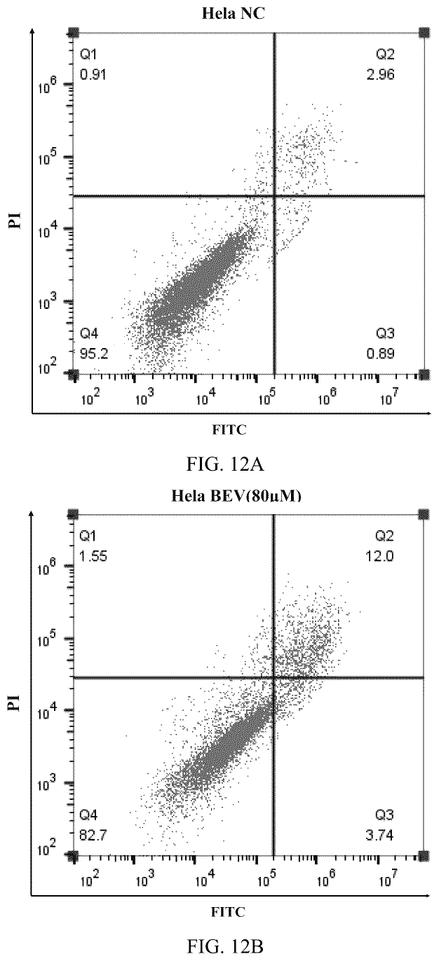

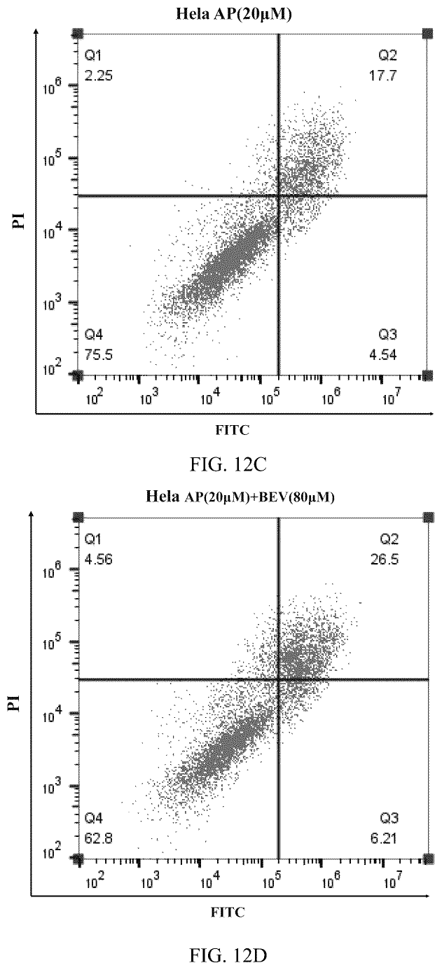

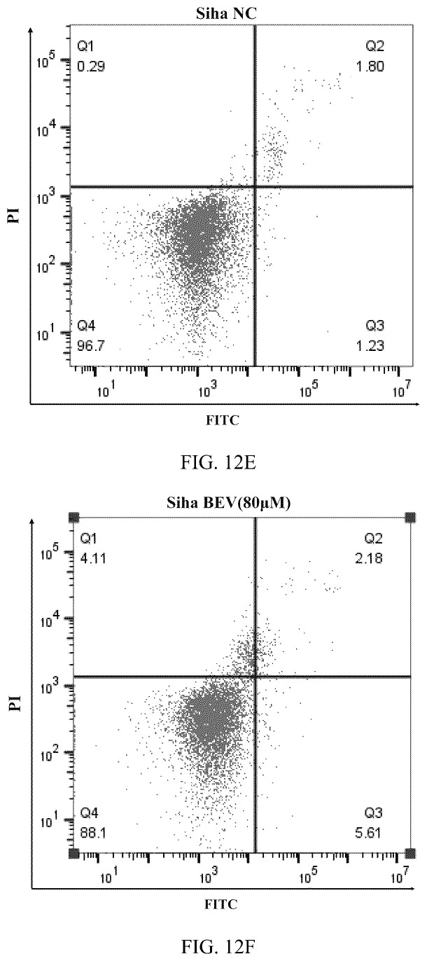

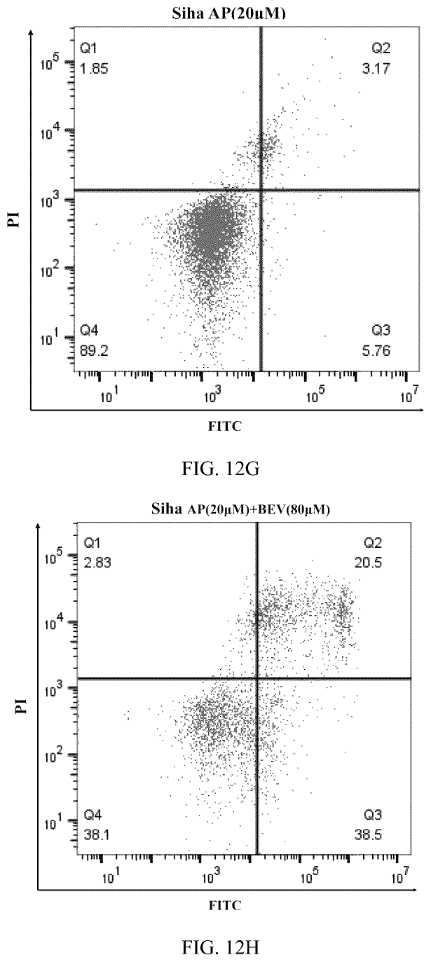

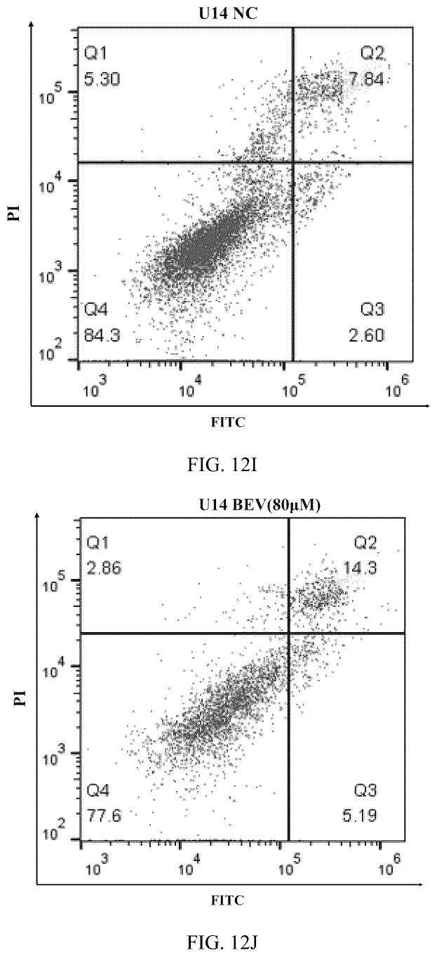

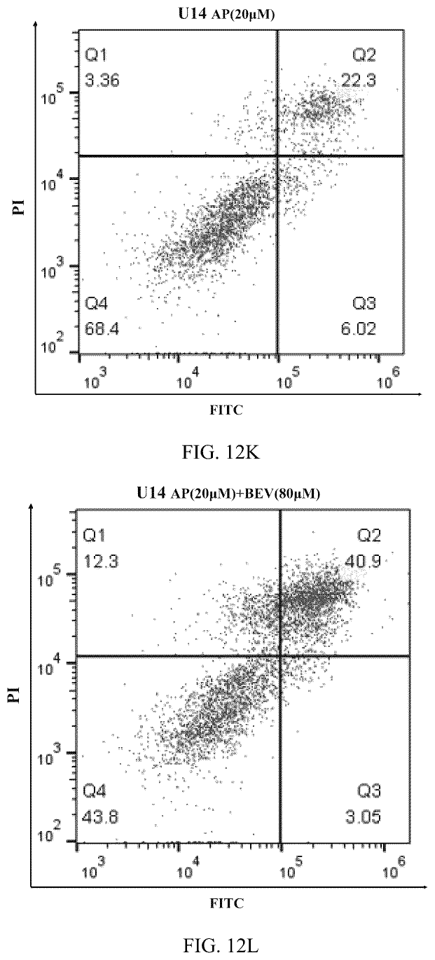

Hela, Siha, and U14 cells in the logarithmic growth phase were digested and centrifuged. After being resuspended, the cells were seeded into 6 cm culture dishes, with 6 dishes spread out. The cells were treated with drugs 24 hours after the cells were completely attached. Drug concentration: The final concentration of Apicidin was 20 μM, and the final concentration of Bevacizumab was 80 μM. The order of drug addition was: blank (NC), Apicidin (Ap), Bevacizumab (Bev), and Apicidin+Bevacizumab (Ap+Bev). After a period of combined drug use, the cells shrank and the lysis rate reached 40%. The next step was performed. Protein extraction: After drug treatment, the supernatant of the cells was discarded, and the cells were washed twice with PBS. An appropriate amount of cell lysis buffer was added, and the cells were lysed on ice for 5 min. The cells were scraped and the lysate was transferred to an EP tube. After lysis on ice for 20 min, the cells were centrifuged at 4° C. and 12000 rpm for 20 minutes, and the supernatant was collected. After the protein concentration was measured in the BCA working method, the supernatant was diluted to 1× by adding 5× protein loading buffer, boiled in a metal bath at 100° C. for 10 min, and then loaded. According to the Western blotting experimental procedures, the expression of apoptosis-related Cleaved-caspase-3, Bax, and Bcl-2 was detected. Apoptosis detection: According to the apoptosis kit operating instructions, 10× Annexin V binding buffer was diluted to 1× Annexin V binding buffer. After drug treatment, the supernatant of the cells was discarded, and the cells were washed twice with PBS, trypsinized, and centrifuged. The supernatant was discarded, and 1 mL of 1× Annexin V binding Buffer was added to resuspend the cells and centrifuged. The supernatant was discarded, and the above steps were repeated once. 400 μL of 1× Annexin V binding Buffer was added to resuspend the cells. Then, 5 μL of FITC/PI reagent was added, and the cells were protected from light for 15 min before detection. The results are shown in FIG. 11 and FIGS. 12A-12L. Compared with the use of either drug alone, the combined use of Apicidin with the targeted drug Bevacizumab significantly increases the expression of apoptosis-related proteins Cleaved-caspase-3 and Bax, significantly decreases the expression of Bcl-2, and significantly increases the number of apoptotic cells. The results suggest that Apicidin can enhance the ability of the targeted drug Bevacizumab to induce apoptosis in cervical cancer cells Hela, Siha, and U14.

### Example 2

### In Vivo Experiments

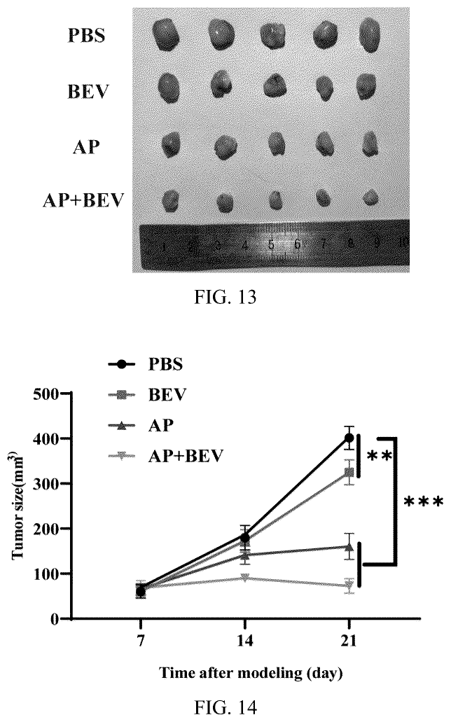

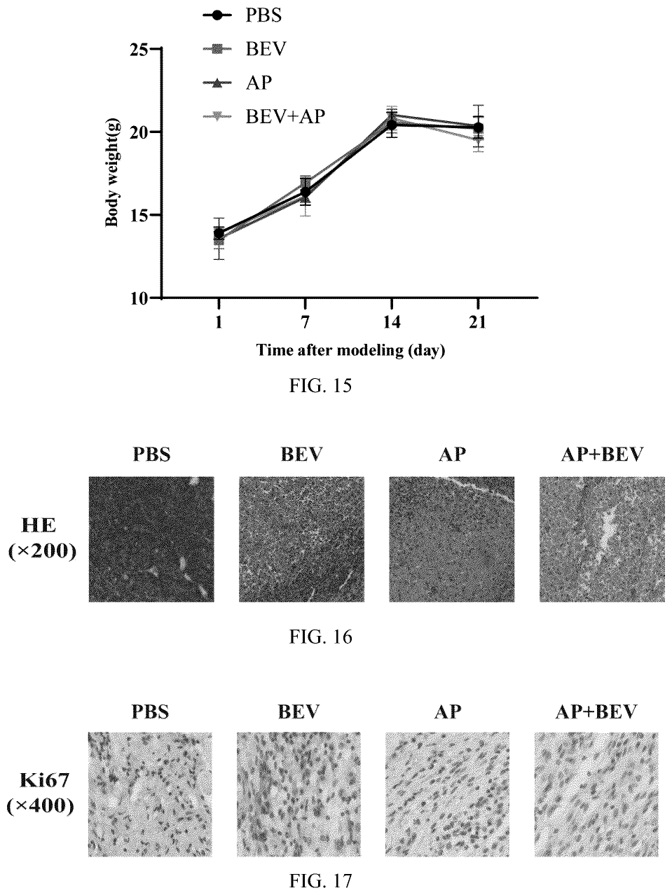

Based on the in vitro experimental results, in vivo functional experiments in nude mice were performed. U14 cells in the logarithmic growth phase were obtained and resuspended in pre-cooled PBS. 1×10⁶/100 μL U14 cells were injected subcutaneously into the right side of the axilla of each mouse. A subcutaneous cervical cancer tumor model was established in mice. The tumor formation in nude mice was observed. When the tumor grew to about 100 mm³, the mice were randomly divided into 4 groups according to the weight and tumor size, with 5 mice in each group. The mice were given drug treatment and divided into the following groups: control group (PBS), Apicidin group (AP), Bevacizumab group (BEV), and Apicidin+Bevacizumab group (AP+BEV). The drug concentrations used were: PBS group (100 μL/day in intraperitoneal injection), Apicidin group (5 mg/kg/2 days in intraperitoneal injection), Bevacizumab group (5 mg/kg/2 days in intraperitoneal injection); Apicidin+Bevacizumab group (5 mg/kg+5 mg/kg/2 days in intraperitoneal injection). The behavioral treatment period was 14 days. After the start of treatment, the length (a) and width (b) of the tumor were measured with a vernier caliper every three days, and the tumor volume was calculated using the specific calculation formula: volume V=0.5 ab². After 15 days of drug treatment, the nude mice showed cachexia such as emaciation. The mice were then killed by spinal dislocation, and the tumor tissues were dissected out and photographed. The corresponding EP tubes were marked and fixed in 4% tissue fixative. The results in FIGS. 13 to 15 show that during the drug treatment of nude mice, the drug group inhibited the weight of nude mice and the growth of subcutaneous tumors compared with the control group. The combined drug group had a stronger ability to inhibit the body weight and subcutaneous tumor growth of nude mice. The results in FIGS. 16 and 17 show that HE staining test indicates that compared with the groups treated with either drug alone, the Apicidin combined with Bevacizumab group has the most severe tumor tissue disintegration or necrotic damage. Immunohistochemistry (IHC) detection indicates that compared with the groups treated with either drug alone, the Apicidin combined with Bevacizumab more effectively reduces the expression of Ki67 in tumor tissue. FIG. 17 shows that in the Ki67 immunohistochemical grayscale image, the nuclear outlines appear light gray. Cells exhibiting dark gray to black granular deposits within the nuclei are classified as Ki67-positive cells. The experimental results show that the nuclear staining in the combined treatment group is light and the positive index is low. The results in FIGS. 13 to 17 indicate that Apicidin inhibits the growth of subcutaneous tumors in nude mice, and the ability of Apicidin combined with Bevacizumab to inhibit tumor growth is enhanced.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A use of Apicidin combined with Bevacizumab in preparing a drug for treating cervical cancer.

2. The use according to claim 1, wherein a molar ratio of a combined use of the Apicidin and the Bevacizumab is (10-40): (10-320).
